(19) United States
(12) Patent Application Publication
(10) Pub. No.: US 2025/0385014 A1
(43) Pub. Date: Dec. 18, 2025

(54) Systems and Methods for Conducting Medical Studies

(71) Applicant: MG Stroke Analytics Inc., Calgary (CA)

(72) Inventors: Mayank Goyal, Calgary (CA); Johanna Ospel, Calgary (CA); Jeffrey Saver, Calgary (CA); Nishita Singh, Calgary (CA); Joachim Christian Fladt, Calgary (CA)

(73) Assignee: MG Stroke Analytics Inc., Calgary (CA)

(21) Appl. No.: 19/130,401

(22) PCT Filed: Nov. 14, 2023
(86) PCT No.: PCT/CA2023/051525

§ 371 Date: May 15, 2025

Related U.S. Application Data
(60) Provisional application No. 63/383,744, filed on Nov. 15, 2022.

(51) Int. Cl. G16H 70/20; A61B 50/30; G16H 10/20; G16H 20/10

(52) U.S. Cl. CPC G16H 70/20; A61B 50/30; G16H 10/20; G16H 20/10

(57) ABSTRACT

Systems and methods for collecting medical data in a pre-hospital setting are described. Modular study devices (MSDs) are comprehensive, customizable toolkits that provide tools/equipment to enable data collection and particularly the tools/equipment to conduct a particular medical study in the pre-hospital setting. Specifically, the equipment and processes allow for standardized data collection and facilitate operating procedures alongside routine clinical care, thereby streamlining study workflow and pre-hospital treatment steps by emergency medical services (EMS) personnel such as paramedics. In addition, systems and methods are described enabling the efficient design of MSDs that may be configured into medical transport vehicles to meet parameters of those vehicles such as volume and/or weight restrictions whilst enabling a customized or unique study protocol to be conducted in the pre-hospital setting.

### FIELD

Systems and methods for collecting medical data in a pre-hospital setting are described. Modular study devices (MSDs) are comprehensive, customizable toolkits that provide tools/equipment to enable data collection and particularly the tools/equipment to conduct a particular medical study in the pre-hospital setting. Specifically, the equipment and processes allow for standardized data collection and facilitate operating procedures alongside routine clinical care, thereby streamlining study workflow and pre-hospital treatment steps by emergency medical services (EMS) personnel such as paramedics. In addition, systems and methods are described enabling the efficient design of MSDs that may be configured into medical transport vehicles to meet parameters of those vehicles such as volume and/or weight restrictions whilst enabling a customized or unique study protocol to be conducted in the pre-hospital setting.

### BACKGROUND

Pre-hospital care is an important part of the healthcare system. Pre-hospital care refers to the acute care that patients receive between the time at which an emergency call is made until hospital arrival and is provided by a variety of emergency care providers such as ambulance and emergency medical services (EMS) personnel (e.g., paramedics) (1,2).

Historically, pre-hospital care focused on swift transport of patients to the nearest emergency department after a quick on-scene assessment (commonly referred to as “load and go”) (3). However, there can be key disadvantages to using this strategy. For example, in acute medical emergencies, time is often critical which essentially means that response to treatment/intervention is highly time dependent. The shorter the time to treatment the higher the chances for good patient outcome. Thus, utilizing the paramedics and ambulance services for primarily transport alone, but not for providing treatment in the field in cases where treatment in the field is possible, prolongs the time to treatment which in turn can increase the odds of having a poor outcome.

As it is a primary objective of health care systems to continually improve patient care, there continues to be a need for systems and methods that improve patient care through earlier and accurate diagnosis and earlier treatment of some time-sensitive medical conditions. In particular, there has been a need for systems that can improve patient care in the pre-hospital setting and specifically when a health care system has been accessed to transport patients to a medical care facility. An important component of improving medical care is conducting medical research to determine if an experimental hypothesis is supported by evidence.

Experimental protocols to conduct medical research are well established in many settings. However, systems and processes to conduct medical research in pre-hospitals settings can be improved.

### SUMMARY

In a first aspect, an apparatus for supporting a research study having a pre-determined research study protocol defined by a sequence of study protocol steps is described wherein the research study protocol is designed for a mobile or remote environment having physical size and/or weight limits for research study equipment. The apparatus includes a container configured for transportation and to contain at least one item of research equipment; at least one associated computer system having at least one processor, and an input and display system, the at least one processor having non-transient memory configured to output to the display system a sequence of the study protocol steps including access to and use of the at least one item of research equipment; and wherein the at least one processor has non-transient memory configured to receive data from the input system.

In various embodiments:
The study protocol includes a study protocol step requiring data input of at least one collectable data parameter related to the study and the processor is configured to receive and store the at least one collectable data parameter during execution of the study protocol.
The study protocol is configured within the associated computer system as a sequence of study steps defining the study protocol and wherein the study protocol includes at least two sub-protocols having a sequence of sub-protocol steps and wherein the combination of sub-protocol steps and study steps collectively define the study protocol.
Each sub-protocol is configured to another sub-protocol and wherein upon completion of one sub-protocol step triggers activation of a sub-protocol step in another sub-protocol.
Steps of different sub-protocols are displayed simultaneously on separate computer systems to different users.
The sub-protocols include a study delivery protocol and at least one of a trial-eligibility, training, diagnosis, consent, randomization, safety, resource allocation, inventory, and documentation sub-protocols.
Upon activation of the study protocol, the display system displays one or more active steps from one or more sub-protocols and wherein a displayed step comprises display of an instruction step.
An instruction step requires data input to proceed to another step.
Based on data input, data is stored and/or reported to the processor and/or a central computer system.
The apparatus includes a communication system associated with the container, the communication system configured to: connect to a wide-area network to deliver to and/or receive data from at least one central computer system; and, receive data from at least one item of research equipment wherein the research equipment is configured to report research data to the communication system during execution of a step of the research study protocol.
The container includes a refrigeration system and refrigeration compartment.
The apparatus includes a power connector within the container, the power connector configured to connect to a vehicle power supply and provide power to at least one item of research equipment within the container, the at least one item of research equipment required for undertaking the pre-determined research study protocol.
The container is configured with a security system, the security system configured to enable authorized access to the container by authorized users.
The security system is configured to connect to the at least one central computer system and report when an authorized user has accessed the container.
The security system includes any one of or a combination of FOBs, keys and/or a code.
The container includes primary and secondary compartments, and the security system includes independent security to access each of the primary and secondary compartments.
The study protocol includes a consent sub-protocol having a plurality of defined steps to prompt for and receive consent/assent to conduct a medical procedure on a patient.
The apparatus includes at least one time display configured to monitor and display time being taken to complete individual or multiple protocol steps.
The apparatus includes any one of or a combination of at least one temperature sensor, pressure sensor, vibration sensor, location sensor, and movement sensor and wherein the processor is configured to receive and store data from each sensor.
The apparatus includes a timer configured to provide a visual and/or audio output of time for at least one step of a study protocol.
The compartment is configured for connection to an emergency vehicle having a pre-determined volume and weight limit for the compartment.

In another aspect, a method for designing a modular study device (MSD) is described, the MSD for supporting a research study having a pre-determined research study protocol defined by a sequence of study protocol steps, the research study protocol having been designed for a mobile or remote environment having physical size and/or weight limits for research study equipment, the method comprising the steps of: a) displaying to and enabling a user to select at least one item of study equipment from a study equipment library wherein the study equipment library includes physical size and/or weight information about each item of study equipment; b) displaying and enabling a user to select a container from a container library wherein the container library includes volume information about each container, each container within the library configured for storage of at least one item of study equipment; c) upon selection of at least one item of study equipment and a container, determining if the at least one item of study equipment selected in step a fits within a container selected in step b and providing output; and, d) determining if the container selected in step b exceeds the physical size and/or weight limits.

In one embodiment, the method includes displaying and enabling a user to select at least one data communications system from a library of data communications systems, wherein the library of data communications systems includes specifications of data acquisition and reporting protocols and study equipment from the library of study equipment that may be configured to each data communication system.

In one embodiment, the method includes the step of displaying and enabling a user to select at least one patient consent protocol from a library of patient consent protocols.

In another aspect, a method of designing a study protocol for a research study is described, where the research study to be conducted is in a mobile or remote environment having physical size and/or weight limits for research study equipment, the study protocol to be defined by a sequence of study protocol steps, the method including the steps of: using a non-transitory computer readable medium encoded with instructions to perform the following steps: a) enabling a user to compile a series of steps defining a sub-protocol and displaying a sub-protocol to a user; b) prompting a user to link one or more steps of a sub-protocol to one or more steps of a different sub-protocol; and, c) prompting a user to configure two or more steps of a sub-protocol to output data to a user and receive physical parameter data related to research equipment as input and wherein the output or input of data completes a step of a sub-protocol; and, wherein upon configuration of two or more sub-protocols, a study protocol is defined having boundary parameters for conducting the study protocol in a mobile environment and wherein the study protocol includes linked steps of a study delivery sub-protocol and any one or more of a trial-eligibility, training, diagnosis, consent, randomization, safety, resource allocation, inventory, and documentation sub-protocols.

In one embodiment, the method includes the step of activating the study protocol on a mobile computer system wherein the mobile computer system is configured to progressively display study protocol steps and upon progression through the study protocol steps, at least one step includes receiving data from related research equipment.

In another embodiment, the method includes the step of configuring at least one step of a sub-protocol of the study protocol to an apparatus as defined herein.

In one embodiment, the study protocol is configured to provide data to two or more physically separated users on separate computer system during execution of the study protocol.

In another aspect, a portable apparatus for treatment of a patient in a pre-hospital setting is described, the apparatus including: a container configured for transportation and to contain at least one item of medical treatment equipment; at least one associated computer system having at least one processor, and an input and display system, the at least one processor having non-transient memory configured to output to the display system a sequence of the treatment protocol steps including access to and use of the at least one item of medical treatment equipment; and wherein the at least one processor has non-transient memory configured to receive data from the input system relevant to a medical treatment protocol and wherein the medical treatment protocol is derived from a research study protocol defined by a sequence of study protocol steps, the research study protocol having been previously designed for a mobile or remote environment having physical size and/or weight limits for research study equipment and wherein the treatment protocol steps are a simplified version of the research study protocol.

In various embodiments:
The treatment protocol includes a treatment protocol step requiring data input of at least one collectable data parameter related to the treatment and the processor is configured to receive and store the at least one collectable data parameter during execution of the treatment protocol.
The treatment protocol includes sub-protocols and each sub-protocol is configured to another sub-protocol and wherein upon completion of one sub-protocol step triggers activation of a sub-protocol step in another sub-protocol.
Steps of different sub-protocols are displayed simultaneously.
The sub-protocols include a treatment delivery protocol and at least one of a training, diagnosis, consent, safety, resource allocation, inventory, and documentation sub-protocols.
Upon activation of the treatment protocol, the display system displays one or more active steps from one or more sub-protocols and wherein a displayed step comprises display of an instruction step.
An instruction step requires data input to proceed to another step.
Data is stored and/or reported to the processor and/or a central computer system.
The apparatus includes a communication system associated with the container, the communication system configured to: connect to a wide-area network to deliver to and/or receive data from at least one central computer system; and, receive data from at least one item of treatment equipment wherein the treatment equipment is configured to report treatment data to the communication system during execution of a step of the treatment protocol.
The container includes a refrigeration system and refrigeration compartment.
The apparatus includes a power connector within the container, the power connector configured to connect to a vehicle power supply and provide power to at least one item of treatment equipment within the container, the at least one item of treatment equipment required for undertaking the pre-determined treatment protocol.
The container is configured with a security system, the security system configured to enable authorized access to the container by authorized users.
The security system is configured to connect to the at least one central computer system and report when an authorized user has accessed the container.
The security system includes any one of or a combination of FOBs, keys and/or a code.
The container includes primary and secondary compartments, and the security system includes independent security to access each of the primary and secondary compartments.
The treatment protocol includes a consent sub-protocol having a plurality of defined steps to prompt for and receive consent/assent to conduct a medical procedure on a patient.
The apparatus includes at least one time display configured to monitor and display time being taken to complete individual or multiple protocol steps.
The apparatus includes any one of or a combination of at least one temperature sensor, pressure sensor, vibration sensor, location sensor, and movement sensor and wherein the processor is configured to receive and store data from each sensor.
The apparatus includes a timer configured to provide a visual and/or audio output of time for at least one step of a treatment protocol.
The compartment is configured for connection to an emergency vehicle having a pre-determined volume and weight limit for the compartment.

### DESCRIPTION OF DRAWINGS

The invention is described with reference to the drawings in which:

FIG. 1 is a schematic overview of a typical ambulance and a communications network in which it may operate in accordance with the prior art.

FIG. 2 is a flowchart showing a generalized pre-hospital randomized study process in accordance with the prior art, using the example of a pharmaceutical study.

FIG. 3 is a flowchart showing a modular study device system that may be deployed in a pre-hospital randomized study setting in accordance with one embodiment of the invention, using the example of a pharmaceutical study.

FIG. 4 is a flowchart showing protocol steps that may be followed in a pre-hospital setting with a modular study device.

FIG. 5 is a representative overview of various functional systems that may be integrated within a modular study device.

FIG. 6 is a representative overview of various components and functional systems that may be integrated within a modular study device.

FIG. 6A is a schematic diagram showing a study protocol and a range of sub-protocols that may be part of a modular study device system and the potential interaction of protocols with one another.

FIG. 7 is flowchart showing a representative series of functional steps that may be activated and/or displayed to users during execution of a study protocol.

FIG. 8 illustrates libraries of data that may be accessed by design software configured to design an MSD.

FIG. 9 is a flowchart of representative steps of a design process that design software may be configured to execute during design of an MSD.

FIG. 10 is a representation of a display system showing active display of sub-protocol steps.

FIG. 11 is a flowchart of representative steps of a protocol displaying steps that may or may not require user input.

FIG. 12 is a schematic diagram showing simplified steps for designing and linking sub-protocols, conducting a study and on study-completion designing a modular treatment kit.

### DETAILED DESCRIPTION

### Overview

Systems and methods to improve processes to diagnose and treat patients whilst conducting medical research in a pre-hospital and/or ex-hospital settings are described. Systems and methods to design a study for a pre- and/or ex-hospital setting are also described.

In the context of this description, a pre-hospital setting is generally defined as an area outside of a healthcare facility. A pre-hospital setting as described herein generally includes a temporal component between the time when a patient may initially engage with a health care system and the time they may receive medical services until arrival at a treatment center. The pre-hospital setting therefore includes various areas or locations including patient homes, any other indoor or outdoor location where a patient may be exhibiting symptoms as well as medical transport vehicles (e.g., ambulances, air-ambulances, fixed-wing aircraft, medical boats, fire vehicles and the like). Pre-hospital setting implies an element of urgency to the extent that emergency vehicles are involved.

An ex-hospital setting is generally defined as an area outside of a healthcare facility but may not include a temporal component that implies urgency. In this context, medical studies may be conducted that do not have an urgent time component but otherwise seek to collect data outside of a medical facility. An example of such a study may be collecting blood data from a patient group that has undergone particular physical stressors (e.g., a high-altitude training exercise) for the purpose of assessing the effects of the physical stresses.

The systems and methods described herein are primarily described in relation to pre-hospital settings but can be applied to ex-hospital settings as well.

By way of background, in situations where a patient engages a health care system and requires transportation to a healthcare facility, a medical transportation vehicle may be dispatched to collect the patient. For the purposes of this description, the medical transportation vehicle will be described as an ambulance, although it is understood that the description applies to other medical transportation vehicles, examples of which are noted above. As known, when a patient exhibits symptoms, a call for emergency services may be initiated. An ambulance is dispatched with trained personnel, mostly emergency medical technicians (EMTs) and/or paramedics, and in some countries also emergency physicians.

EMTs are typically trained in basic life support care, including performing cardiopulmonary resuscitation and administering oxygen with the main goals of the paramedics being to stabilize patients and provide safe, monitored transport to the hospital.

Paramedics are typically trained in both basic and advanced life support care and can perform more complex procedures like inserting IV lines and administering drugs. The leading goals of EMTs and paramedics are to stabilize patients and provide safe, monitored transport to the hospital.

The paramedic scope of practice typically includes a circumscribed set of early treatment interventions, such as and including cardioversion, cardiac pacing, hemorrhage control, and intramuscular/subcutaneous injections, and intravenous push injections, though generally not controlled intravenous infusions via infusion pumps (8). Many current and future treatments for medical emergencies like acute stroke, myocardial infarction, status epilepticus and trauma have a large magnitude of benefit with faster treatment but are currently out of paramedic scope of practice (4,5).

As known, medical technology and diagnostic and treatment protocols are ever-evolving wherein new technologies could provide benefits to patients including the pre-hospital setting. Advances in medical technology include the development of diagnostic and/or treatment technologies. These may include new devices, drugs, protocols and the like that may improve patient care.

While such technological progress may be of advantage in the pre-hospital setting, testing new technologies in pre-hospital settings can be difficult due to a wide range of factors including physical factors and human factors. Physical factors may include volume and weight considerations of equipment within an ambulance, the portability of equipment, the sensitivity of equipment to vibration or movement and/or efficient collection and utilization of medical data. Human factors may include the complexity of operation of new equipment and/or training of personnel to operate the equipment or conduct new procedures.

Overcoming these issues is desirable, particularly when it is believed that testing new technologies in the pre-hospital phase would allow for accurate, systematic evaluation of the added value of these technologies. If a technology has been shown to add value, it could be adopted and both refine clinical evaluation and diagnosis and improve management of patients on-scene and during transport as well as improving diagnosis and treatment at the hospital, all of which may lead to a better outcome.

Similarly, as part of the process of leading to better health care, it is also important to identify technologies that are not of added value if the data indicates no definitive benefit.

Scientific research aims to close knowledge gaps by building the evidence base for optimized standards of patient care. Despite increasing efforts over the last decades to conduct pre-hospital studies, the body of evidence in many sub-areas of prehospital care is lacking behind the level of evidence for the in-hospital setting (6) as a result of the problems identified above, resulting in relatively few pre-hospital studies having been conducted. Thus, there is relatively little experience in conducting scientific research in the pre-hospital setting (7), and only a few prehospital treatment options besides basic and advanced life support, patient stabilization and monitoring have found their way into clinical routine.

In addition, and in furtherance to the above, some of the reasons why only few pre-hospital studies are conducted are practical barriers such as:
a. lack of storage space and accessibility of specific study materials in ordinary EMS vehicles;
b. configuring study equipment to be transportable and usable within the limited space of ordinary EMS vehicles;
c. lack of physical presence of study personnel in the field;
d. lack of “manpower” (too few paramedics to allow for study-related tasks in addition to clinical care);
e. difficulties securing compliance to study protocols given different work practices, skill and knowledge levels among paramedics;
f. difficulties in obtaining consent to therapies or study recruitment from acutely ill patients in emergency situations;
g. short time period available to perform study activities;
h. costs;
i. barriers to entry including where the overall process is felt to be too complex and onerous;
j. cultural barriers;
k. difficulty in identifying patients that could be suitable for the study;
l. lack of design tools to enable researchers to efficiently design study-protocols and study equipment for use within the pre-hospital setting.

Furthermore, upon completion of a pre-hospital study, there is a need to effectively transition equipment/procedures that were shown to be of benefit by the study to a usable package of equipment/procedures as a new standard of care for pre-hospital diagnosis and/or treatment.

For researchers/trialists, overcoming such barriers and developing pre-hospital strategies that fulfill the specific needs of their trial is a challenge that hampers implementation of research and can prevent further progress in pre-hospital care.

### Overview-Systems and Methods for Pre-Hospital Based Medical Research

In view of the foregoing, there is a need for systems, apparatuses and methods that enable investigators who want to conduct pre-hospital studies to design and configure a modular study device (MSD) according to specific study requirements which minimizes or solves some or all of the aforementioned problems.

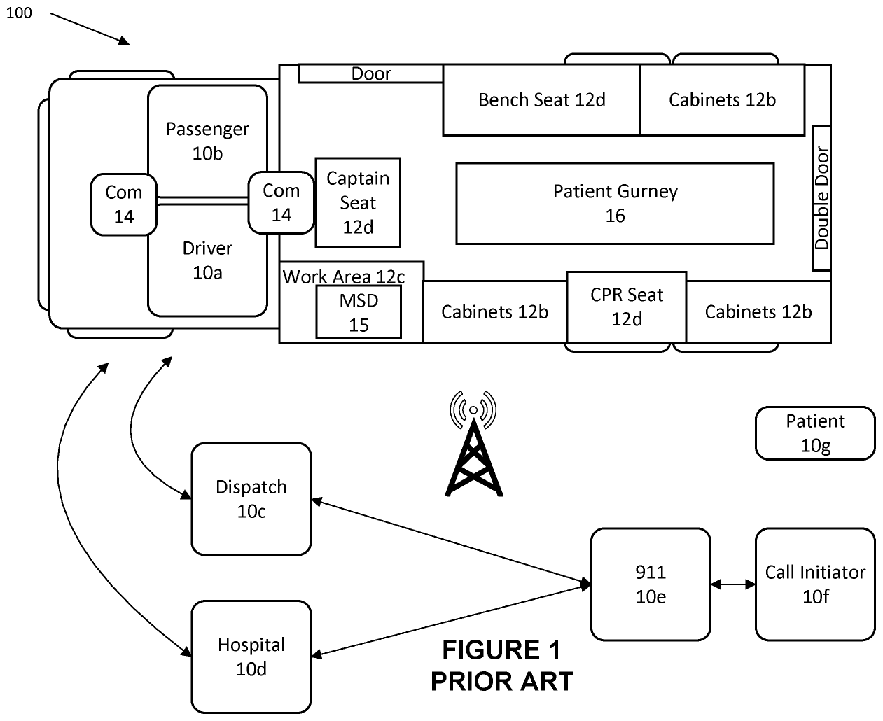

To provide context to the systems, apparatuses and methods described herein, a typical ambulance is described together with typical deployment scenarios. With reference to FIG. 1, a representative ambulance is shown with typical/standard equipment that may be configured within an ambulance as well as a representative communications network that enables the ambulance to operate within the catchment area of the ambulance. The catchment area of an ambulance may be within a defined jurisdiction and be part of a wide area health care system that may typically extend up to a few hundred kilometers from one or more care centers depending on location.

A typical ambulance operates with an ambulance team whose personnel includes at least a driver 10a and at least one attending first-responder 10b. The ambulance team engages with various parties at different times, including for example, a dispatcher 10c, a hospital 10d, an emergency dispatcher (e.g., 911 or 999 emergency call system) 10e, a call initiator 10f (e.g., a family member or a by-stander) and a patient 10g.

An emergency call may be initiated by a patient/call initiator 10f/10g to an emergency dispatcher 10e such as a 911 dispatcher. As a result of talking with the initiator, the 911 dispatcher 10e may deploy an ambulance 100 directly or through a separate ambulance dispatcher 10c. The ambulance dispatcher 10c or 911 operator 911 may communicate with the ambulance team 10a/10b with destination instructions and the anticipated nature of the call. The ambulance dispatcher will typically also contact the care facility 10d (e.g., hospital) where it may be initially contemplated that the patient may be taken. On-going communications between the patient/call initiator, 911 service, and hospital (and various combinations thereof) may be initiated and/or continued where additional information about the nature of the patient's condition is obtained during the call depending on the nature of the medical situation.

As shown in FIG. 1, ambulances are typically kitted with a patient gurney 16 for loading the patient into the ambulance and securing them for transport. The ambulance has a wide range of medical equipment available within various compartments 12b to provide advanced first aid and advanced life support (ALS) as well as various work areas 12c and seating 12d that may be utilized to prepare and/or configure equipment. Equipment may include various computers and electronics with various displays that are connected to standard and/or specialized diagnostic/monitoring equipment. Importantly, all medical equipment that is kitted within an ambulance must carefully consider the ability for the equipment to be stored efficiently within the ambulance and to otherwise be usable by personnel. Equipment that is overly heavy, bulky, sensitive to movement/vibration, temperatures, etc. will not be deployed in an ambulance.

The ambulance may also be kitted with a range of communications systems 14 that enable real-time communication with dispatchers, the destination hospital and its physicians and specialist physicians located within or outside the hospital. Location data through GPS may be available and reported in real-time to the dispatchers and hospital. Dispatch systems may also implement coded messaging in the form of specialized codes and/or texts to communicate data to/from ambulances.

For example, during a call, the ambulance team may have been provided by the connected people (e.g., dispatcher, patient and/or bystanders) with ongoing communications about the condition and symptoms of the patient to enable them to prepare themselves and the equipment that may be needed on their arrival.

Depending on the information received, status information may be directed to the ambulance team, such that upon arrival they have prepared equipment such as automated external defibrillators (AEDs), blood pressure cuffs, intravenous lines, etc., for prompt use upon arrival.

It is also noted that prior to a call being initiated, in the background, additional personnel and systems are in place to ensure that the ambulance is ready and configured to meet a wide range of potential medical conditions. This includes many support services including vehicle maintenance and cleaning, updating and managing inventory, replacing expired medication and medical devices, personnel management, personnel training and so on.

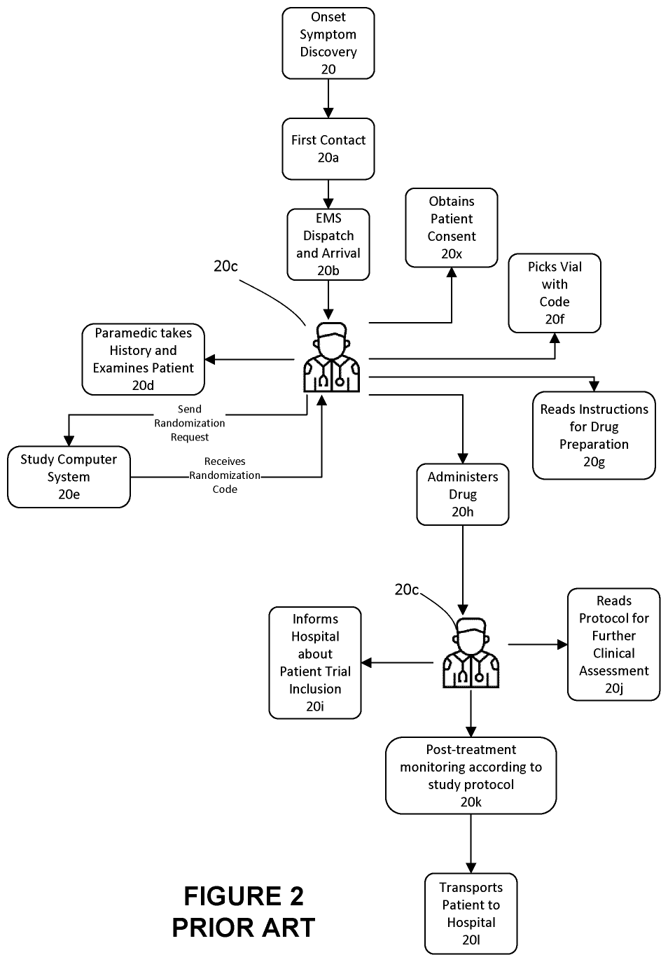

Currently, as shown in FIG. 2, if an ambulance has been outfitted and its personnel trained to conduct a pre-hospital study involving testing a new drug, the following steps are typically followed.

A patient may exhibit symptoms 20 and a person makes contact 20a with an ambulance dispatcher. The ambulance is dispatched and travels to the patient 20b. Upon arrival, the paramedic 20c takes the patient's history and otherwise examines the patient 20d. If the paramedic has been trained for the study and believes the patient may be eligible (e.g. for a study testing a medication or an investigational drug), the paramedic may obtain patient consent 20x and send a request to a central system 20e indicating a suitable patient and requesting randomization information. The central system may then send information on allocation of the study arm that the patient has been randomized back to the paramedic that is read by the paramedic as instructions to select a specific vial with a code within the ambulance to administer (the code may reflect a vial number which unknown to the paramedic could either be a drug or a placebo). The paramedic accesses a vial with that code 20f, reads the administration instructions 20g and then administers the drug 20h. The paramedic may then separately inform the hospital of the patient trial inclusion 20i, then read a protocol for further clinical assessment 20j and conduct any post-treatment monitoring 20k before/during transport to the hospital 20l, if needed as per the study protocol.

### Modular Study Device

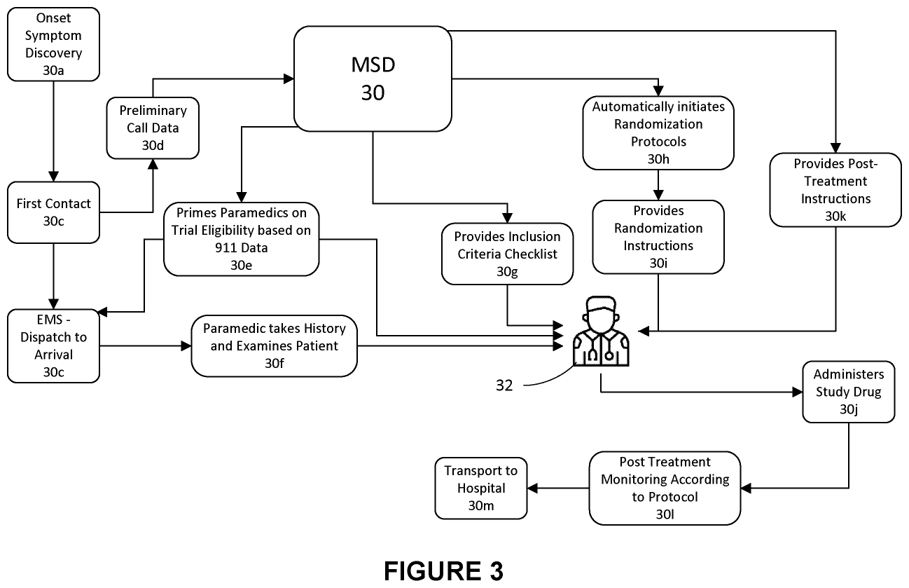

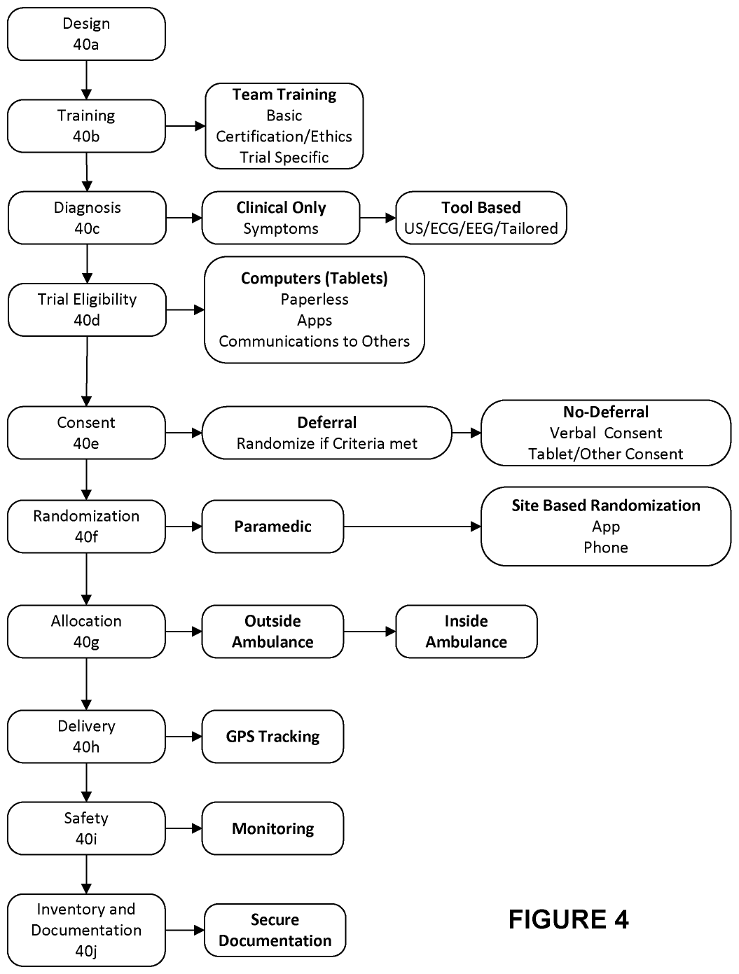

In accordance with one aspect of the invention, a modular study device system and modular study device (MSD) are described that enables a) a principal investigator (or related parties) to design customized MSDs for conducting pre-hospital studies and b) that allow medical personnel described above to conduct pre-hospital studies. In various embodiments, the MSDs described herein ensure the needs of a study and the portability requirements of an ambulance are met. In one embodiment, an MSD is configured to enable personnel to undertake representative steps outlined in FIGS. 3 and 4.

As shown in FIG. 3, in various embodiments a configured MSD 30 can improve the steps of conducting a pre-hospital study as compared to the process outlined in FIG. 2. As shown, a patient may exhibit symptoms 30a and a person makes contact 30b with an ambulance dispatcher. The ambulance is dispatched and travels 30c to the patient with a paramedic 32.

An MSD may be configured to obtain preliminary data from the dispatcher. For example, during dispatch, a dispatcher may enter a dispatch code indicating the suspected nature of the patient's condition. For example, dispatchers may routinely enter codes/text that are transmitted to the ambulance indicating that the patient is suspected of having suffered a stroke or heart attack. This data code/text may be transmitted/communicated 30d with the MSD which can be used to trigger the MSD to activate procedures to start a pre-hospital study protocol. For example, the MSD may be configured to conduct a study related to stroke in which case if the ambulance dispatcher enters a “stroke” code into the dispatch system, the MSD would receive notice of a potentially suitable patient for the study.

If activated, the MSD may take steps to notify the paramedics and provide information 30e to the paramedics regarding aspects of the study. Such information may depend on the nature of the study and could include a checklist of inclusion and exclusion criteria, as well as notification of high level and/or specific protocol steps of the study. Such steps may include preparing equipment or drug(s) and/or displaying information to the paramedics about steps of the study. As such, the paramedic may arrive at the patient in a better-prepared state to execute study steps.

In addition, for studies that require arrival on scene of specialized prehospital personnel (such as nurses or physicians) or of specialized equipment (such as mobile stroke ambulances equipped with CT scanners, mobile lung support ambulances equipped with extracorporeal membrane oxygen devices, mobile accident extrication ambulances equipped to perform field amputation), the MSD may concurrently notify the nearest prehospital specialty personnel and specialized ambulance to proceed to the scene.

Upon arrival, the standard ambulance paramedic 32 would take the patient history and otherwise examine the patient 30f. If the paramedic confirms that the suspected diagnosis is correct, the MSD may be triggered (manually or automatically) to display study inclusion criteria 30g. Once the paramedic has confirmed that the inclusion criteria are met (for example, via voice or manual input into the MSD), such information is stored by the MSD for the study record. Once this is done, the randomization process will be either manually or automatically initiated, and further, instructions may be displayed/communicated 30i to the paramedic resulting from the randomization determination. To ensure use of the correct randomly assigned treatment (for example, treatment option A vs treatment option B), the MSD may selectively unlock the storage container just for the allocated treatment. The paramedic may then administer a study drug 30j (in the case of a pharmacological study).

The MSD 30 may further display/communicate post treatment instructions 30k. The paramedic may also provide post-treatment care 30l while the patient is being transported to the hospital 30m.

In another example, additional functionality may be included in an MSD system as shown in FIG. 4 including front end design 40a and training 40b, protocols for obtaining patient consent 40e, safety protocols 40i and inventory and documentation 40j.

In this example, the MSD system as shown in FIG. 4, provides tools to the researcher to assist the researcher to design a study 40a and to establish training protocols 40b to be followed to collect data for the study. The training protocols are practiced by the study team by using an MSD to guide the team through the protocol, and may include team training modules, basic training, certification, ethics and/or trial specific training.

Once the MSD systems have been used to design an MSD that serves the purpose of the study a particular researcher wants to conduct, and upon successful completion of all training modules by the study team, the study can start enrolment. The MSD can continue to play a role during the actual enrolment period as well, as explained below.

In one embodiment, the MSD is activated at a time when the pre-hospital team's diagnosis 40c suggests a suitable situation. Upon an initial diagnosis, the MSD may determine if the patient meets eligibility criteria 40d for participation in the study. Diagnosis may be based on clinical assessment of the paramedics and/or aided by diagnostic equipment.

If it is determined that a patient is eligible for a study 40d, various steps may be taken to begin record keeping as required by the study through various means including electronic systems including laptops, tablets, apps, etc.

In most studies, obtaining consent 40e will be required. Consent may be recorded by the MSD in different ways depending on the form of consent (e.g., signatures may be stored or audio recordings may be stored in case of oral consent). Depending on the situation, consent may also be deferred or may be obtained by various means.

In some situations, consent may be obtained by various means from the patient or an on-scene legally authorized representative (LAR). When the patient is not competent to provide consent and no LAR is on-scene, attempts to obtain assent may be required. (Assent is agreement to participate in the study to the constrained degree the individual can understand the research and what it means to participate.)

If a patient is eligible, randomization criteria 40f that may be part of the overall study protocol may be implemented in accordance with various procedures.

Study resources 40g may be allocated and delivered/activated 40h for the study within the ambulance and/or may be fully or partially completed outside the ambulance.

In various embodiments, once the study resources have been used, they may be put back into the MSD in a dedicated sterile container for disposal after hospital arrival.

At the appropriate time, the patient is transported 40h to the hospital.

At all times, appropriate safety monitoring 40i protocols and/or inventory and documentation procedures 40j may be followed including GPS tracking, monitoring and secure documentation.

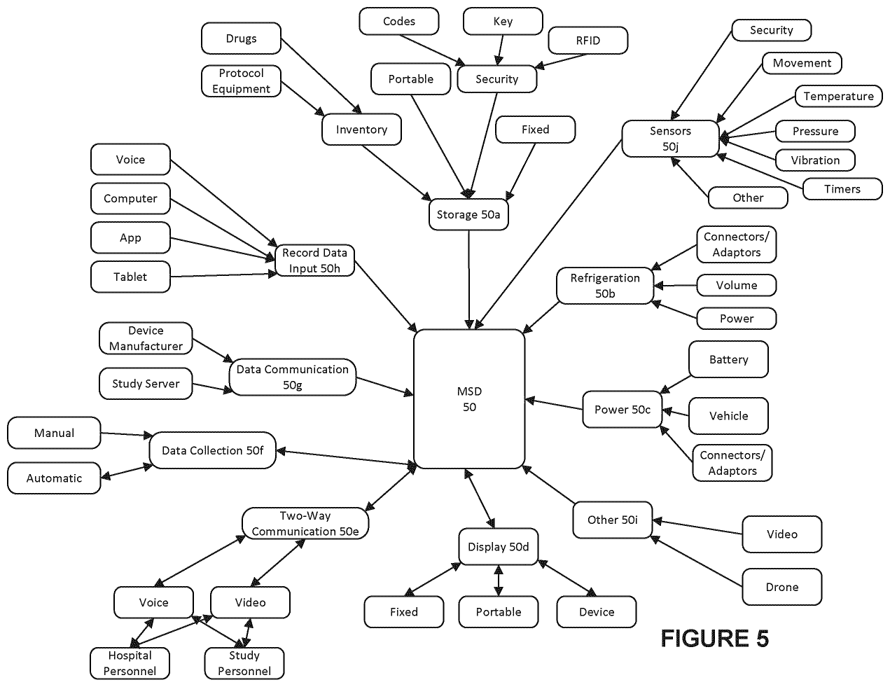

Further details and embodiments of an MSD system are described with reference to FIGS. 5-9. For the purpose of illustration, FIGS. 5-9 are described with reference to representative studies including a study looking to investigate a proposed procedure termed remote ischemic conditioning (RIC). RIC is a proposed study looking to examine if patient outcome of patients suspected of having suffered ischemic stroke can be improved. While aspects of the MSD system and MSD are described with reference to RIC, the principles of designing an MSD system and MSD may be applied to a wide range of studies.

### RIC Study Example

A researcher hypothesizes that occluding blood flow to the lower limbs of a patient suspected of suffering from ischemic stroke during transportation to a hospital improves patient outcome. The researcher has postulated that reducing the volume of circulating blood during transport enriches oxygen saturation of the circulating blood in the other areas of the body and that increased oxygen saturation in the blood may enhance oxygen uptake in ischemic areas of the brain and slow brain death in affected areas. The researcher proposes to have ambulance personnel attach one or two large blood pressure cuffs to the legs of the patient to constrict blood flow to the lower limbs from the time of collection to delivery to the hospital. The study looks to examine parameters of this proposed treatment in combination with additional data collected at the hospital.

The researcher plans the study to collect the following data from the time of attaching the blood pressure cuff(s) to delivery to the hospital:
a. Blood oxygen saturation levels each minute from the time of collection to delivery to the hospital from the non-occluded areas (e.g., from finger tips);
b. Blood oxygen saturation levels each minute from the time of collection to delivery to the hospital from the occluded areas (e.g., from toes);
c. Blood pressure every 5 minutes from the time of collection to delivery to the hospital;
d. Total time that the blood pressure cuffs are attached.

The foregoing data may subsequently be correlated to in-hospital treatment and allow the researcher to determine if ambulance treatment results in a better patient outcome for different severities of ischemic stroke.

In order to implement the study, the researcher initially designs a multi-faceted protocol for the study which includes the following general steps:
a. Process/parameters to diagnose, determine study eligibility and trigger study treatment protocol
b. Process/protocol to follow if step a) parameters are met including processes to:
   i. Obtain patient/LAR consent/assent
   ii. Randomization
c. Design of an MSD for inclusion in an ambulance
d. Training of Health Care workers for study implementation
e. Data Collection Equipment and Protocols
f. Safety Monitoring
g. Data Confirmation and Data Protection

As shown in FIG. 5, the design of an effective MSD 50 can include a wide range of equipment that is brought together and that enables the logical and efficient completion of numerous steps during the study.

During the design of an MSD, the investigator may draw upon a wide range of potential components that may be required for the study. As shown in FIG. 5, these may include storage components/systems 50a (including inventory and security), refrigeration systems 50b, power systems 50c, display systems 50d, two-way communications systems 50e, data collection systems 50f, data communication systems 50g, data input systems 50h, sensor systems 50j and other systems 50i. Each of these MSD systems can include specific types of component systems that may be integrated into an MSD 50. Various MSD systems may include sub-systems and are described in greater detail below.

### Study Protocol, Design and Deployment Using an MSD

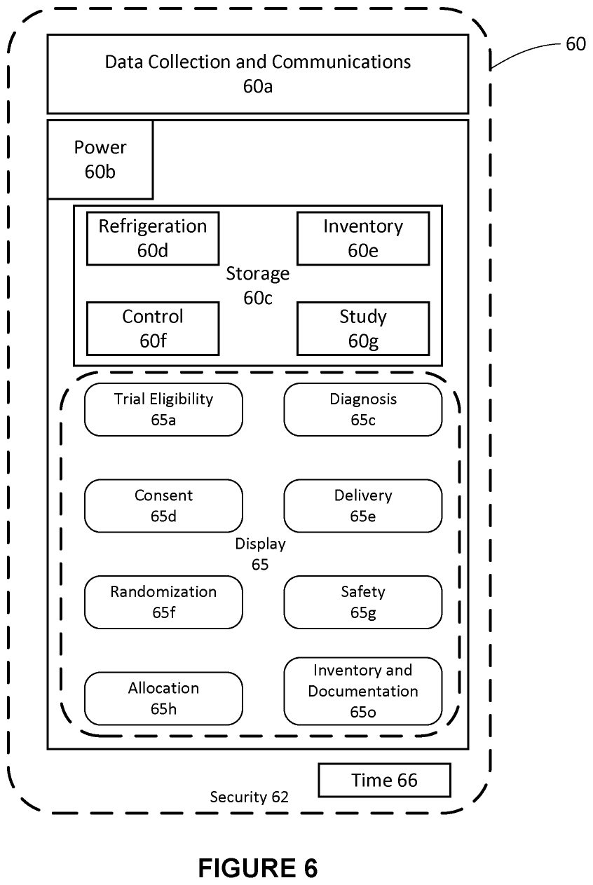

The steps for the design and execution of an example study are described with reference to FIGS. 6 and 6A. FIG. 6 shows an example of an MSD 60 composed of various physical modules including a physical storage container 60c, a data collection and communications module 60a, and a power module 60b. The storage module 60c may include storage areas including a refrigeration compartment 60d, an inventory compartment 60e, and/or specific equipment 60f, 60g for control protocols and study protocols. The MSD may also include various display systems 65 used for displaying study protocols including trial-eligibility 65a, diagnosis 65c, consent/assent 65d, delivery 65e (i.e. patient interaction protocol), randomization 65f, safety 65g, allocation 65h and inventory and documentation 65i protocols.

The MSD may also include one or more integrated time displays 66 configured to keep track of elapsed time for an entire protocol or aspects of a protocol. That is, for various studies it may be important to ensure that steps of the study are completed in accordance with strict time frames. In some embodiments, as part of the MSD design process (described in greater detail below), the MSD and MSD protocols may be configured to provide audio/visual signals to users regarding time limits to complete particular steps. Timers may be started automatically and/or triggered by user input into the MSD.

Some or all of the various MSD modules may be within a security system 62.

Generally, the design of a study protocol includes the general steps of defining study parameters, defining the various protocols that are required to undertake the study and designing an MSD capable of assisting in the execution of defined study protocols.

As shown, a specific study protocol 65 may include sub-protocols that collectively define the scope and/or boundary conditions/parameters of a study. Such sub-protocols may include trial-eligibility 65a, training 65b, diagnosis 65c, consent/assent 65d, delivery 65e (i.e. patient interaction protocol), randomization 65f, safety 65g, allocation 65h and inventory and documentation 65i protocols as shown in FIG. 6A.

Protocols 65 may include a series of independently operating modules that define specific steps to be executed that display, transmit or obtain information to/from users/equipment. Protocols may interact or interface with other protocols that, for example, trigger initiation of another protocol when a step in one protocol is completed, provide data to another protocol and/or interact with components of an MSD including medical equipment and/or security systems 60a.

Protocols may be computer-executable code stored in computer memory with corresponding input devices (e.g., a keyboard, touchscreen, mouse, buttons, various sensors, microphones and the like) and output devices (e.g., monitors, lights, audio, vibratory devices and the like) that assist one or more users in following logical steps to obtain study data in the pre-hospital setting whilst providing appropriate patient care.

A trial eligibility protocol 65a may have the general functionality of assisting users in determining if a patient is eligible for a study at a first or preliminary level.

A training protocol 65b may have the general functionality of providing information to users including paramedics and technicians that describes the purpose, scope and practical steps of execution of a particular study.

A diagnosis protocol 65c may have the general functionality of assisting a user in making a diagnosis of a particular patient's condition to refine a determination of whether a patient is eligible for a study.

A consent/assent protocol 65d may have the general functionality of assisting a user in obtaining consent/assent from a patient/LAR according to jurisdictional and/or ethical requirements.

A delivery/treatment protocol 65e may have the general functionality of assisting a user in conducting steps to complete a medical procedure including for example, use of one or more medical devices, administering a drug(s), completing a number of physical steps/manipulations, etc. A delivery/treatment protocol may include separate protocols for study and control groups.

A randomization protocol 65f may have the general functionality of randomizing patient selection for control and study group protocols.

A safety protocol 65g may have the general functionality of assisting a user in conducting steps of the study protocol in a manner that meets appropriate safety standards for a particular study including the use of particular medical equipment within particular operating conditions.

An allocation protocol 65h may have the general functionality of assisting a user in conducting steps to allocate study resources as may be appropriate for a particular study.

An inventory/documentation protocol 65i may have the general functionality of assisting users in managing study resources within a location or MSD and/or provide documentation and tracking of study resources.

As shown in FIG. 6A, depending on implementation, various sub-protocols may be standalone to the extent that a protocol displays/provides steps to a user to complete or protocols and/or may be connected/interfaced with other protocols to activate other protocols upon certain steps of another protocol having been completed. For example, fulfillment of certain criteria (e.g., the patient is suspected of having suffered a stroke) may automatically trigger activation/display of a diagnosis protocol that displays additional steps to confirm a diagnosis and/or take into account additional factors/data that may confirm a patient's suitability or not for participation in the study. Once a patient has been identified as potentially being eligible for a study, completion of particular steps of trial eligibility protocol may follow. Information may flow bi-directionally between different protocols and may be displayed to a user sequentially or simultaneously.

It is understood that different protocols may or may not be relevant to some studies. In various embodiments, the practical creation and deployment of various protocols may overlap with other protocols where in various examples, data is exchanged between different protocols to trigger display of further steps in another protocol.

In the RIC example, a researcher is interested in conducting a study of patients who are suspected of having suffered ischemic stroke of a particular severity and, if criteria are met, test a RIC procedure. The researcher understands that a range of parameters will define the scope of the study and that the parameters defining the study will ideally enable the collection of data that will test the study hypothesis. The study protocol may ultimately include sub-protocols, each of which may work in conjunction with one another as described above.

In one example, the researcher may wish to implement a “trial eligibility” protocol that will exclude patients that do not meet a particular threshold of severity or are above a particular threshold of severity. The trial-eligibility protocol may also limit the patient population to those patients within a particular age range, weight, and/or take into consideration other parameters that may exclude certain patients for safety and other relevant reasons. Protocols may obtain input data from associated equipment including basic equipment such as body temperature sensors and blood pressure systems or more sophisticated diagnostic/monitoring equipment.

In the RIC example, the study may be designed to collect data from patients who are suspected as suffering a stroke of “mild” to “moderate” severity, who are between 18-80 years of age and are greater than 45 kg in weight. As noted, other parameters may be important.

Based on the desired parameters, the researcher can design a “diagnosis” and “delivery” protocol to be followed by the paramedics. For example, a particular diagnostic protocol may be designed that outlines steps to be followed to make a preliminary diagnosis. In a representative example, these may include conducting a number of preliminary tests such as speech slur, hand grip strength, leg weakness, facial droop, etc. The “trial eligibility” protocol may state that if one or more thresholds are reached, the patient is eligible to participate in the study. As part of the design process, and as described in greater detail below, as each step of the protocol is formulated, various inputs and outputs may be configured to one or more steps that require user input to proceed, and/or data input from other users and/or equipment.

The study protocol may require that other parameters are met, for example requiring that the patient eligibility protocol is initiated only if the patient is more than a predetermined number of minutes/hours away from the hospital as may be determined by GPS/mapping software/data that is available to an MSD.

The study protocol may prompt the personnel to activate study protocol software to ensure that data collection and data records are initiated/created in accordance with any “documentation” protocols or such protocols may be automatically initiated if various conditions are met within other protocols.

In one example, the study protocol may require that for any suspected stroke a patient record is opened prior to determining if the patient does in fact meet the study criteria. If the study protocol subsequently determines that study criteria have not been met, and no further data is collected, this data may also be useful to the study and may be kept as a screening log.

Activation of one or more protocols at an early stage in the overall study protocol may be useful to provide appropriate information and guidance to personnel throughout the call. In one example, the protocol software may provide visual and/or audio prompts to personnel to ensure that steps are properly followed. In one example, the study protocol software may be activated based on information provided by a 911 dispatcher or ambulance dispatch system, wherein the ambulance personnel manually trigger the protocol software on their way to the call or the protocol software is automatically triggered by information received by a dispatch system. During travel to the call, personnel may be advised by recorded voice of the protocol steps and/or protocol steps may be displayed through the protocol software. This can provide continuous training to personnel as well as providing consistency to the execution of the protocol.

If the patient meets study criteria, the protocol may initiate other protocols including patient/LAR consent/assent and randomization protocols.

The study protocol may require that if a patient is determined as eligible, a consent protocol may be activated with steps to obtain patient/LAR consent/assent. A consent protocol may outline steps to obtain patient/LAR consent/assent according to appropriate legal and ethical standards for a particular study. In various embodiments, a consent protocol may require obtaining consent by video, verbally and/or by written consent. The consent protocol may include steps that provide an indication of possible risks. Risks may be presented to the patient via a video and/or app-based presentation. Under various protocols, consent may be obtained from the patient or a legally assigned representative.

Upon completion of the consent protocol, if implemented, the study protocol may also include a randomization protocol. The randomization protocol may include steps to be followed to reduce various biases that could affect study data.

In one example, 50% of eligible patients may receive the study treatment and 50% may receive a control group treatment. The randomization protocol may include automatic randomization as may be determined by study software or by other criteria. The randomization protocol may include steps that provide codes to personnel that determine the steps to be taken or communicate/display particular steps to be followed.

In the RIC example, the study protocol may require that the treatment group be treated according to the treatment protocol which includes applying two large blood pressure cuffs to the lower limbs and attaching blood oxygen meters to one or two of the patient's toes and to a finger and monitoring data until removal. A regular blood pressure cuff may be applied to the patient's arm. The treatment protocol may set an inflation pressure for the large cuffs that will be applied and a maximum time for inflation until release and a time period for auto-activation of the arm cuff.

The treatment protocol may require that the control group be treated according to the control group protocol. In this example, the cuffs and oxygen meters may be applied as with the treatment group, but the inflation pressure is set at a lower level for the leg cuffs. All other data may be collected as per the study protocol.

The treatment protocol may be designed to require that personnel complete the study or control group protocol. Different protocols will require varying degrees of personnel input during protocol execution. In some studies, after equipment is configured and activated, no additional input may be required from personnel in that study equipment may operate automatically and collect and report data automatically.

Other study protocols may require additional input from personnel as part of the study protocol ranging from periodic checks/adjustments to equipment to regular and repeated steps being undertaken.

Various steps may include administration of drugs, equipment placement and adjustment, patient manipulation and any number of protocol specific steps.

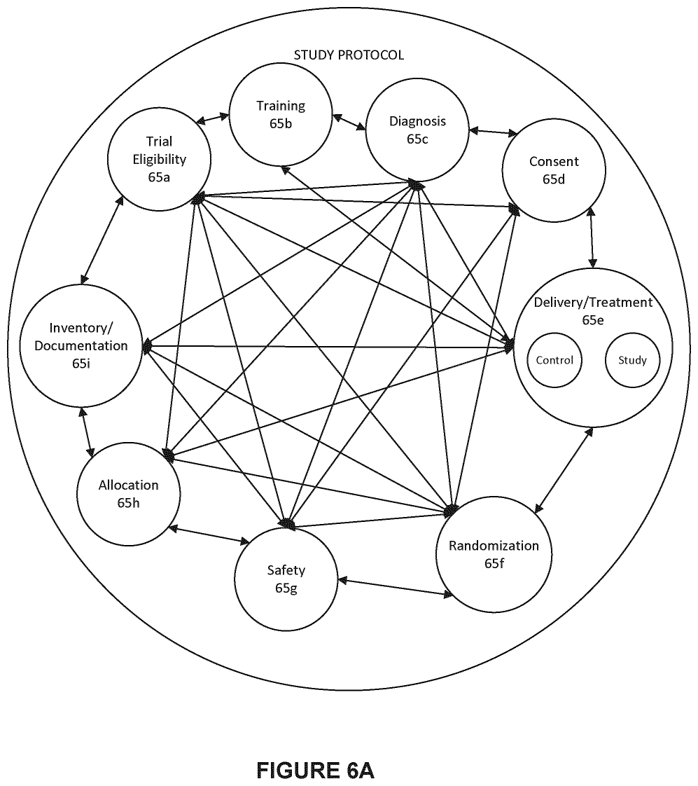

FIG. 7 is a flowchart showing representative steps of study protocol software.

In this example, an MSD has been configured with a computer system that is operable to support the study protocol and sub-protocols. The MSD is a lockable cabinet inside an ambulance that contains study equipment/resources.

The study software is initially in a standby mode 70a. If configured, the software may conduct an inventory check and/or an equipment status check 70b. For example, the MSD may include sensors that monitor refrigeration temperatures wherein if refrigeration temperatures are properly maintained, the software reports “in specification inventory”. The MSD may include sensors that monitor vehicle vibrations during ground transport wherein if vibrations remain in tolerance for study devices, the software reports “in specification inventory”. Similarly, battery operated equipment may have battery charge levels monitored.

If inventory criteria are not met, this may be reported 70c.

If inventory criteria are met, the software may remain in standby until activated by personnel or by other triggers when a preliminary diagnosis 70d is made. Upon activation of the study protocol 70e, various steps may be undertaken according to the study protocol and specifics of the configured MSD. Such steps may include creating a patient file 70f, activating the communication system(s) 70g, displaying the study protocol overview 70h and displaying the consent protocol 70i.

If consent/assent is a required component of the study, personnel may trigger and/or interface with the consent protocol 70i. If consent/assent is received 70j, the software may move to further steps including displaying details of the study protocol 70k. If consent/assent is not received or other factors may require termination of the study protocol (e.g. the patient does not meet study criteria), the system may return to standby.

If the study protocol requires randomization, randomization criteria, (i.e. study inclusion criteria) may be applied 70l. This may be displayed to the personnel or may be automatic as determined by back-end programming and/or may be determined by the inventory. For example, in the case of a drug study, inventory stocking in the MSD may have randomly stocked the study drug(s) or placebo.

Depending on the randomization determination 70m, the software may display different protocols to the personnel including the study group procedure 70n or control group procedure 70o if different. The software may selectively unlock the storage container just for the allocated treatment (among treatment A vs treatment B). In either case, additional information may be displayed to the personnel to access study resources 70p,q and complete the appropriate protocols 70r, s. Data may be collected, stored and reported 70t. The personnel may or may not be aware that a study or control group is being followed.

As noted above, after an MSD system and MSD has been designed, configured and tested for deployment, prior to deployment in an ambulance, ambulance personnel may be trained on the protocols for a particular study. Other personnel may also be trained including hospital and/or equipment stakeholders.

Team training may include visual aids such as PowerPoint presentations or study-specific graphical aids and may be external to the MSD. Team training materials may be deployed and accessible within the MSD for reference when an ambulance team has down-time and wishes to review materials.

### MSD Design Process and Design Configuration System and Software (DCS)

In accordance with another aspect, systems and processes for designing an MSD are described including application software that is configured to enable an effective MSD to be built.

As shown in FIGS. 5, 6, 6A and 8, an MSD may be designed by enabling a researcher to select equipment and modules that may be incorporated into an MSD for a study to be conducted. Modules may be physical modules involving collections/assemblies of physical equipment or may be computer modules including executable code that is integrated into a computer/communications system and that may be integrated to interact with other physical or computer modules. Executable code may include templates of sub-protocols that define various steps and that may be configurable by editing software to define specific steps and/or integrate with other protocols and/or hardware.

As noted above, as there are a number of limitations in the assembly of an MSD including available volume, power and weight, available equipment, available communications systems and other limitations, the configuration of an MSD potentially requires trade-offs in design to ensure that the MSD is usable within an ambulance.

Design configuration software (DCS) generally operates with a plurality of data libraries enabling a designer to select key features of an MSD including the storage components, the equipment that may be configured within a storage component, communications systems, security systems, sensors, data control systems as well as the protocols and sub-protocols. The design configuration software may be configured to consider limitations that may be imposed on an MSD design by the volume space, weight, power, etc. limitations for a specific ambulance and/or fleet of ambulances.

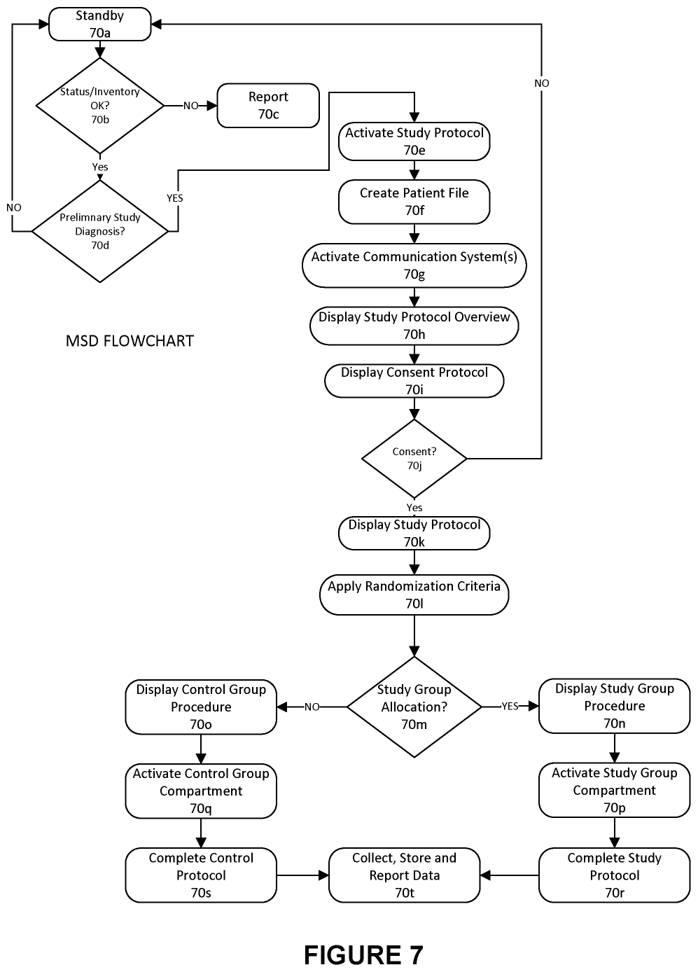

As shown in FIG. 8, in one representative example, the design configuration software to design an MSD 80 may be configured to display/enable a number of libraries allowing the design of an MSD system and MSD. These libraries may include an ambulance library 80a, a container library 80b, an equipment library 80c, a security library 80d, a data control library 80e, a diagnosis/treatment library 80f, a consent/assent library 80g, a communications library 80h, a sensor library 80j and other libraries 80i, any or all of which may be appropriate for a study. Each library will generally be a database of available equipment and/or procedures.

A protocol within a protocol library may be a template outlining a series of steps that includes steps of accessing, activating and configuring general/specific equipment that may be edited for a specific study. Different protocol templates may be appropriate for the general type of study (e.g., equipment, drug, data-only) and various sub-classes of studies within a general type.

If the study protocol requires the use of equipment, the equipment library may enable a user to access and review equipment and the parameters of that equipment to determine if that equipment is appropriate for a particular study.

For example, a proposed study may propose the use of a blood pressure cuff as part of the study protocol and it is desirable to obtain blood pressure information every 3 minutes during the study. There are numerous blood pressure systems on the market, ranging from fully manual to fully automatic with a range of control functions and reporting functionalities. Some cuffs may be readily configured to existing data systems and automatically obtain and report data whereas others only store data that must be manually downloaded to a specific type of computer. Some cuffs may be configured to report data to the cloud via Bluetooth/Wi-Fi if in close proximity to a computer system such as a tablet with specific application software and/or Wi-Fi network.

As such, based on the library of available cuffs, the designer may be able to select a cuff that is most suitable to the proposed protocol. In one instance, real-time data may be required by the study in order to make various decisions whereas in another study, delayed data is sufficient. By comparing available parameters, the designer is able to select a device that may be most suitable for their study.

Similarly, other parameters may be evaluated including the weight and volume of the proposed equipment. A particular piece of equipment may be substantially larger than competitor equipment in which case, storage volume could be an issue. Other important parameters including operating temperatures, sensitivity to movement, etc. may be important for a particular study.

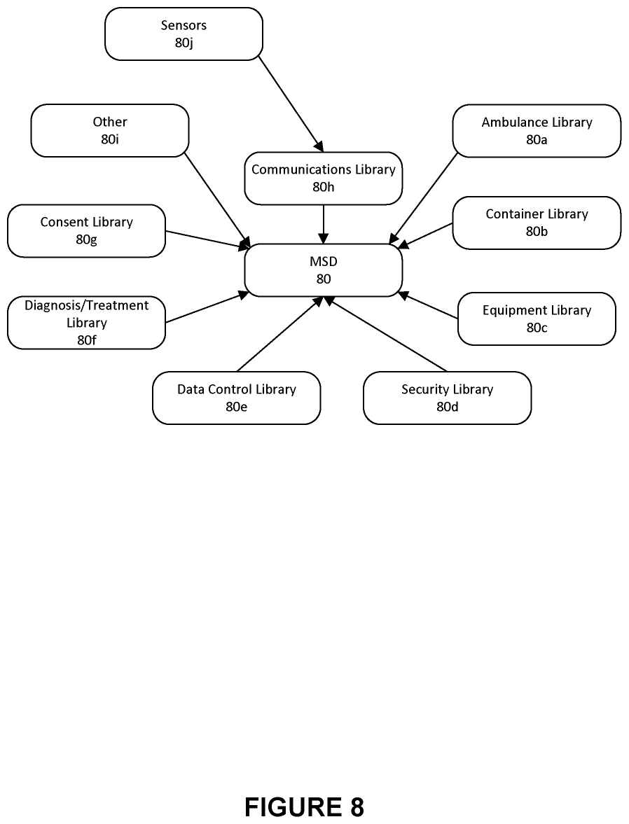

The design configuration software may enable a designer to select equipment for the study and keep a running total of the combined and individual weights and volumes of selected equipment (and/or other parameters) as shown in FIG. 9.

For example, an investigator/designer may have designed a preliminary protocol 90 that requires selection of equipment to be included in the study. Available equipment can be displayed 90a and the investigator can select equipment 90b.

From the selected equipment, a running total of weights and volumes can be calculated 90c, 90d and used as a basis for which to select a particular container 90e. For example, the proposed assortment of study equipment may have a combined volume of 10 liters and 5 kg which could be successfully contained within 5 different “standard” containers within the container library.

Hence, a designer could then select and review each of these containers from the container library 90f to determine if the standard containers met other criteria. For example, 3 standard containers may include refrigeration systems that would not be required in which case, they could be discarded as suitable for the study. In addition, one container may have the volume but is soft-sided and would not meet the security requirements for the study and thus be discarded.

In addition, an ambulance library 90g may also be displayed and/or analyzed 90i to determine if the selected container may be configurable within a particular ambulance/ambulance fleet.

Similarly other libraries as described above may be accessed to refine and test a design.

Generally, if a preliminary match 90h is made, it may be tested against other parameters including for example, the ability to be configured within a particular ambulance 90i. If problems 90j are found, problem parameters may be identified allowing the design to modify the equipment 90k or amend a protocol 90l.

During this process, an ethics review 90m may be required.

Custom containers 90n may be required.

In one embodiment, upon initial confirmation of a potentially acceptable MSD design 90i, the DCS may further conduct time modelling 90p of the anticipated times to complete the study protocol. In this embodiment, a user may input time values of key steps in order to model the time to complete a protocol. Modelled times (and/or ranges of times) may be compared to acceptable times to determine if a protocol can be reasonably completed within a desired time frame 90q. If the modelled times are too long, the DCS may prompt the designer to check protocol steps 90l to determine if there is a better way to execute steps to ensure an acceptable time.

If the DCS confirms that physical design parameters are acceptable, an MSD design may be finalized 90o. Upon the design of a MSD system that meets physical and time parameters, the final design may be added to a study design library 90r.

It is understood that FIG. 9 is merely illustrative of one particular process that may be followed and that the MSD design as a whole can be enabled for a designer to gain access to all available libraries and match equipment, containers, communications, power, security, data collection, etc. and provide feedback to the designer such that sophisticated study MSDs can be physically configured.

In addition, the design configuration software may also be configured to enable the designer to select other study components including consent protocols. That is, consent protocols may be specific to the type of study wherein required consent/assent may be appropriate to the type of study. Consent protocols may be required to take into account specifics of a particular jurisdiction; hence, the library of consent protocols may be efficiently used to assist in setting up the study in one region based on previously written consent protocols.

In various embodiments, the design configuration software may include additional modules and/or libraries and/or functionality to assist the designer configuring an MSD for preliminary testing and/or deployment. Preliminary design configurations may be finalized and then built and be subjected to preliminary testing. Preliminary testing may confirm the effectiveness of a design or may identify issues that could be problematic in the field.

If problems are identified as noted above, the designer may return to the DCS and activate the DCS to update a design.

In various embodiments, as introduced above, the DCS may be configured to enable a designer to choose a “base” MSD depending on the type of study being conducted and/or enable access to previously designed studies/systems. Different types of studies may be conducted in an ambulance including:
1. Medical Device Study
   a. Researching effectiveness of a new medical device
2. Drug Study
   a. Researching effectiveness of new drug treatment including new drugs, dosages, combinations, etc.
3. Treatment Study
   a. Researching effectiveness of a treatment protocol, etc.
4. Diagnostic study
   a. Researching novel blood tests or device tests (e.g., ultrasound, EEG, infrared) to recognize a disease as present and/or quantify disease severity, etc.
5. Observational Study
   a. Research involving ongoing data collection in a specific population to study natural history in the pre-hospital phase.

Each of these general types of studies will typically require different collections of equipment for the study. For example, researching a new medical device (e.g., a new automated chest compression device) to deliver chest compressions to a patient without a pulse will require a different collection of equipment compared to a drug study, where a new drug requires both refrigeration, dosage control equipment and specific data/security protocols.

The common functionality of two MSDs for the above examples may be a lockable cabinet, power and communications systems that provides real-time or substantially real-time communications capabilities with one or more central computer systems and storage space for protocol equipment.

### MSD Features

Referring back to FIG. 5, as noted an MSD 50 may include a range of systems required for successful deployment in the field including within an ambulance. Various features of an MSD are listed which may be configured in various combinations:
lockable cabinet having a size suitable for storage in an ambulance
configurable to existing anchoring systems
attached to existing power supplies and communications systems within the ambulance
self-contained
independent battery power source
independent communications systems
hard case that is permanently or semi-permanently connected to the ambulance and/or a soft case that is designed for portability or a combination thereof
multiple storage components that are separated and stored in spatial proximity and/or configured to separate locations in the ambulance
Security system(s) having a range of layers including codes, keys, RFID, two-person validation
Sensors configured to monitor any number of variable or data input types
central automatic documentation and auditing
two-way communication including audio, video
etc.

The MSD may be designed to be distinguishable from other standard containers/compartments that may be present in an ambulance including distinct labeling that clearly identifies study equipment as being part of the study.

The MSD may be designed to withstand certain conditions as required by the local circumstances and/or applications, such as being configured for carrying (e.g., as a backpack), being waterproof, and/or light-weight for different applications, including maritime, air-borne or field applications.

### Security

The MSD may be configured with a security system having one or more layers of security. Security may be implemented for various reasons and to meet various safety and/or regulatory requirements. Basic security may be implemented as a means of ensuring only trained/authorized personnel are given access to the MSD. That is, a first ambulance team may have been trained to conduct a protocol but a second team who works in the same ambulance or fleet of ambulances has not. The first ambulance team may be provided with access via various security systems such as security codes, keys, RFID tags, fingerprint ID, retina scanners, facial recognition or the like whereas the second team may not.

Security may be implemented at various levels and have different layers of security within a designed MPSTD. For example, a refrigerator within the MPSTD may only be opened by personnel having a 2^{nd} and higher level of security to gain access to drugs within the refrigerator whereas a first level of security is only required to gain access to other study materials in the other compartment(s) of the MPSTD.

### Refrigeration

Refrigeration may be required for various studies. As above, refrigeration requires a volume as well as other parameters including power load and/or the need for adaptors to connect to power sources including ambulance power and/or backup power.

Refrigeration systems may have higher or lower power requirements based on the volume and operating temperatures and/or their own power systems including batteries.

### Power

Generally, the overall power requirements may be calculated and compared to the known ambulance power supply. As above, to the extent that a researcher is designing a study utilizing equipment that may have power requirements that exceed the available power of an ambulance, the researcher may have to re-design the study and/or look to utilizing different equipment that can meet the power restrictions.

### Communications

Numerous communications systems may be configured depending on requirements including one or two-way data communication and/or voice and/or video communication.

In one example, video communication could be multichannel to allow the offsite team to be able to see the patient (and device) from various angles e.g., to view patient's facial expressions and look for asymmetry in case of suspected stroke or e.g., to see from various angles that the study device has been installed properly on the patient e.g., an automatic chest compression device.

### Display

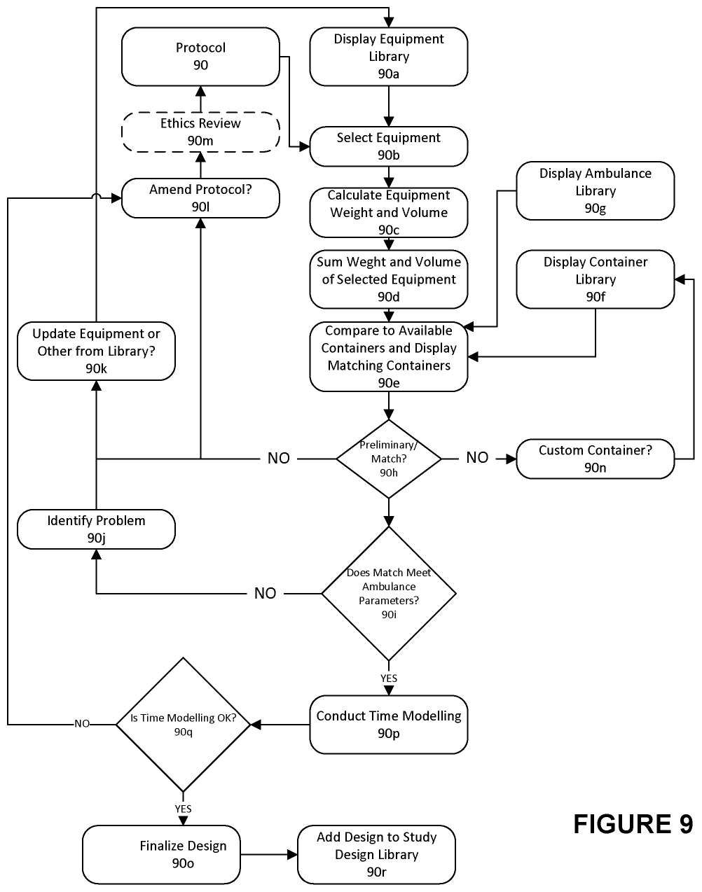

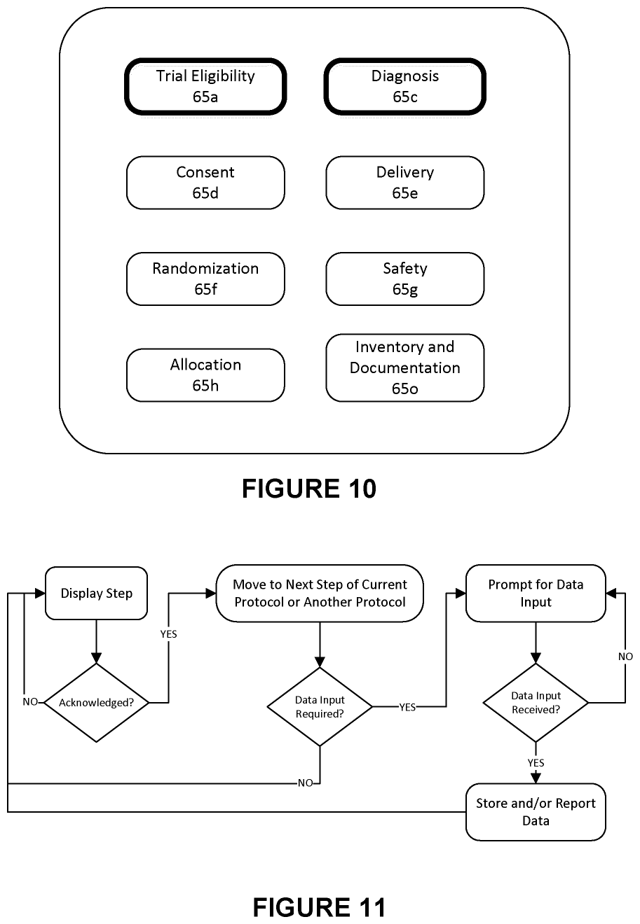

With reference to FIGS. 10 and 11, in various embodiments information that is displayed to users is organized to ensure that the steps of the study are undertaken accurately within the intended boundaries of the study to ensure that experimental error are minimized. As such, following steps as presented to a user in logical sequence can minimize the risk of steps being omitted and/or being conducted in a manner that increases the likelihood of experimental error.

As shown in FIG. 10, a display system may visually display some or all of the sub-protocols of a study in a manner that ensures a user knows where they are in the overall protocol. Current step(s) being followed may be displayed prominently such that the user can readily interact with those step(s).

As shown in FIG. 11, steps may be configured in a manner that sequentially displays a step to the user. Depending on the system configuration, the step may be acknowledged by the user before the next step is displayed. A step may require data input that may prompt a user to manually enter data and/or data may be collected automatically from associated medical equipment if so configured. Data that is collected may be stored and/or reported to a computer system before the next step in the protocol is displayed.

It is understood that a wide range of user/equipment interactions may be configured to the system so as to efficiently and accurately collect data.

### Sensors

Numerous sensors or sensor systems may form part of an MSD to collect a range of data and data types. Examples include temperature, pressure, vibration, time, location, movement, security sensors and others.

### Protocol Design, Study Completion and Subsequent Kit Design

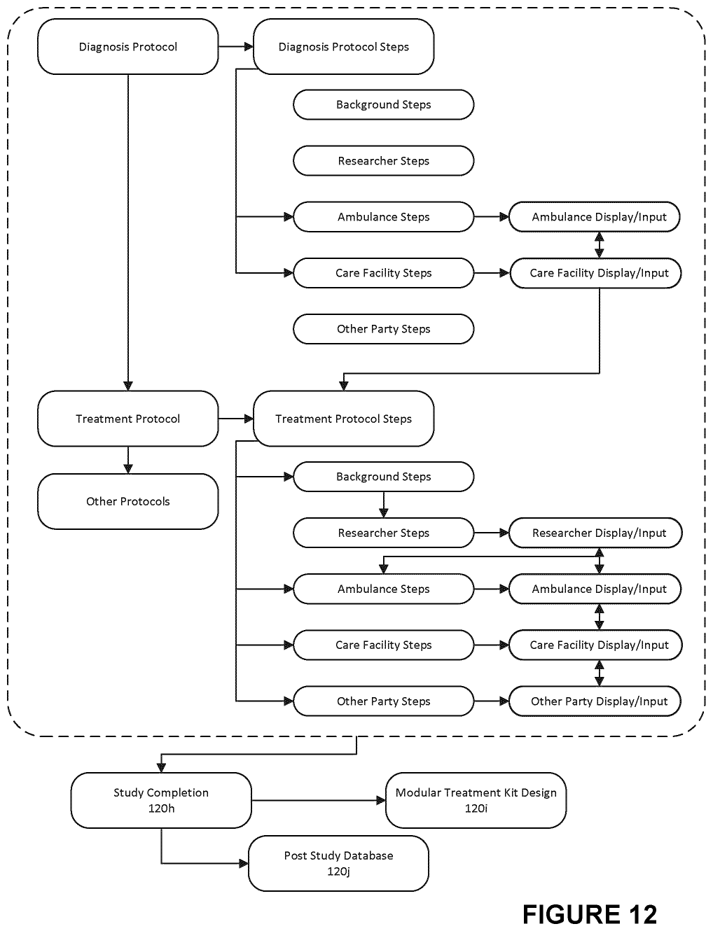

As shown in FIG. 12, in one embodiment, a study protocol 120 may be designed with links between a series of sub-protocols such as those shown in FIG. 6A. In one embodiment, the design and inter-connection of different sub-protocols can be implemented by a designer in a manner that efficiently enables a designer to display and interconnect protocol steps from different protocols to design an overall study protocol for a particular study.

FIG. 12 is a schematic diagram illustrating how sub-protocols may be displayed and linked to one another such that stakeholders (for example, a researcher, ambulance personnel, care facility personnel and other parties) that may be part of the study protocol may be interconnected within the overall protocol.

By way of example, a particular study protocol may include a diagnosis sub-protocol 120 that could involve different stakeholders to effect an accurate diagnosis. Stakeholders could include the researcher 120b, ambulance personnel 120b, care facility personnel 120d and other parties 120e. A number of steps may also be background steps that do not require input/display from/to stakeholders.

During the design phase, the study designer may design the diagnosis protocol such that the ambulance personnel execute particular steps to make a diagnostic determination. In an example, particular information may be displayed to them that may require various inputs 120f as outlined in FIG. 11 in order to proceed. During the design process, the designer may create a number of steps that require involvement of the ambulance personnel as well as care facility personnel 120d, who may be required to provide input to confirm/assist 120g in a diagnosis. That is, the protocol would be set up such that certain steps are displayed to ambulance personnel to prompt them to perform one or more steps (e.g., attach a device to a patient and transmit data to the care facility). These particular steps may be configured with a series of options from one or more drop-down lists or other data entry formats that enable the creation of links/prompts etc. to various stakeholders with the objective of creating a network of interconnected displays, prompts, links and the like to create the overall workflow.

The links between stakeholders in either the same sub-protocol or another sub-protocols may also require incorporation of appropriate logical operators and/or programming to design an efficient work flow that supports consistency and accuracy.

For example, the diagnosis protocol may have 10 steps with 5 steps performed by ambulance personnel and 5 steps performed care facility personnel. Step 2 of 5 of the ambulance personnel steps may be to contact care facility personnel to obtain input.

In various embodiments, it is understood that the system is set-up to enable the designer who may be researcher, to establish a generalized scheme for the study protocol that may not inherently be fully functional as a fully configured and activated MSD. Rather, in various embodiments, the general flow of steps may be configured by the designer but may require detailed programming to enable hardware to communicate within various components of the MSD.

Ultimately, upon completion of the design process, which may include testing to verify that hardware and software is properly communicating, the MSD can be activated in accordance with the activation steps of the protocol.

Upon completion of a study, which may have involved deployment of a single, a few or a large number of MSDs in the field to collect sufficient data, the results of study will be analyzed to determine if the study hypothesis has been proven or not. If the study is successful, incorporation of a new drug, treatment, or medical device, etc. into regular pre-hospital care may be desired.

Generally, in many studies, the steps of the study protocol would represent the majority of the steps for a new pre-hospital treatment. However, in most cases, some steps may be removed and/or be adjusted to become a new treatment protocol.

Typically, this may involve removing a few steps from one or more protocols, that may not be relevant to treatment post-study and configuring a modular treatment kit (MTK) for use within an ambulance. An MTK may be substantially the same as an MSD but may also be simpler and/or involve different equipment. For example, to the extent that evolution to an MTK does not include control group randomization and treatment protocols for study and control groups, placebos, and/or the same security, data acquisition and communication systems, the MTK system may be simpler and be smaller in weight and volume.

Accordingly, in various embodiments, upon completion of a study, the study designer may access the design software and system and follow similar steps to remove and/or de-link aspects of the study protocol that are not relevant to an MTK system.

Similarly, the design software may also be used to design an MTK that utilizes similar but different components as outlined in FIG. 5. Components such as the data acquisition systems, data display, audio/video systems, communication systems, storage systems, etc. may be replaced with smaller, less sophisticated equipment to the extent that the level of detail within an MTK may not be required.

Upon completion of a study, the study protocol and the study results may be uploaded to a post study database 120j as an accessible body of information for other researchers to review for the design of similar studies.

### Scope

The foregoing discussion provides many example embodiments. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Although the present invention has been described and illustrated with respect to preferred embodiments and preferred uses thereof, it is not to be so limited since modifications and changes can be made therein which are within the full, intended scope of the invention as understood by those skilled in the art.

### References

1. Bigham B L, Bull E, Morrison M et al. Patient safety in emergency medical services: executive summary and recommendations from the Niagara Summit. Can J Emerg Med 2011;13:13-8.

2. Farlex Medical Dictionary for the Health Professions and Nursing. Feasterville, PA: Farlex Partner Medical Dictionary Inc, 2012).

3. The Ems Examination Task Force American Board Of Emergency M, Perina D G, Pons P T, Blackwell T H, Bogucki S, Brice J H, et al. The core content of emergency medical services medicine. Prehosp Emerg Care. 2012; 16:309-22.

4. Emberson J,et al; Stroke Thrombolysis Trialists' Collaborative Group. Effect of treatment delay, age, and stroke severity on the effects of intravenous thrombolysis with alteplase for acute ischemic stroke: a meta-analysis of individual patient data from randomised trials. Lancet. 2014 Nov. 29;384(9958):1929-35.

5. Hagiwara M, Bremer A, Claesson A, Axelsson C, Norberg G, Herlitz J. The impact of direct admission to a catheterisation lab/CCU in patients with ST-elevation myocardial infarction on the delay to reperfusion and early risk of death: results of a systematic review including meta-analysis. Scand J Trauma Resusc Emerg Med. 2014;22:67.

6. Turner J. Building the evidence base in pre-hospital urgent and emergency care: a review of research evidence and priorities for future research. Department of Health; 2010.

7. Pocock H. et al; Human factors in prehospital research: lessons from the PARAMEDIC trial

8. National Association of State EMS Officials. (2021 August). National EMS scope of practice model 2019: Including change notices 1.0 and 2.0 (Report No. DOT HS 813 151). National Highway Traffic Safety Administration.

1. An apparatus for supporting a research study having a pre-determined research study protocol defined by a sequence of study protocol steps, the research study protocol having been designed for a mobile or remote environment having physical size and/or weight limits for research study equipment, comprising:
a container configured for transportation and to contain at least one item of research equipment;
at least one associated computer system having at least one processor, and an input and display system,
the at least one processor having non-transient memory configured to output to the display system a sequence of the study protocol steps including access to and use of the at least one item of research equipment; and,
and wherein the at least one processor has non-transient memory configured to receive data from the input system.

2. The apparatus as in claim 1 wherein the study protocol includes a study protocol step requiring data input of at least one collectable data parameter related to the study and the processor is configured to receive and store the at least one collectable data parameter during execution of the study protocol.

3. The apparatus as in claim 1 wherein the study protocol is configured within the associated computer system as a sequence of study steps defining the study protocol and wherein the study protocol includes at least two sub-protocols having a sequence of sub-protocol steps and wherein the combination of sub-protocol steps and study steps collectively define the study protocol.

4. The apparatus as in claim 1 wherein each sub-protocol is configured to another sub-protocol and wherein upon completion of one sub-protocol step triggers activation of a sub-protocol step in another sub-protocol.

5. The apparatus as in claim 1 wherein steps of different sub-protocols are displayed simultaneously.

6. The apparatus as in claim 1 wherein steps of different sub-protocols are displayed simultaneously on separate computer systems to different users.

7. The apparatus as in claim 3 wherein the sub-protocols include a study delivery protocol and at least one of a trial-eligibility, training, diagnosis, consent, randomization, safety, resource allocation, inventory, and documentation sub-protocols.

8. The apparatus as in claim 1 wherein, upon activation of the study protocol, the display system displays one or more active steps from one or more sub-protocols and wherein a displayed step comprises display of an instruction step.

9. The apparatus as in claim 8 wherein an instruction step requires data input to proceed to another step.

10. The apparatus as in claim 7 wherein, based on data input, data is stored and/or reported to the processor and/or a central computer system.

11. The apparatus as in claim 1 further comprising a communication system associated with the container, the communication system configured to:
connect to a wide-area network to deliver to and/or receive data from at least one central computer system; and,
receive data from at least one item of research equipment wherein the research equipment is configured to report research data to the communication system during execution of a step of the research study protocol.

12. The apparatus as in claim 1 wherein the container includes a refrigeration system and refrigeration compartment.

13. The apparatus as in claim 1 further comprising a power connector within the container, the power connector configured to connect to a vehicle power supply and provide power to at least one item of research equipment within the container, the at least one item of research equipment required for undertaking the pre-determined research study protocol.

14. The apparatus as in claim 1 wherein the container is configured with a security system, the security system configured to enable authorized access to the container by authorized users.

15. The apparatus as in claim 14 where the security system is configured to connect to the at least one central computer system and report when an authorized user has accessed the container.

16. The apparatus as in claim 14 where the security system includes any one of or a combination of FOBs, keys and/or a code.

17. The apparatus as in claim 14 wherein the container includes primary and secondary compartments, and the security system includes independent security to access each of the primary and secondary compartments.

18. The apparatus as in claim 1 wherein the study protocol includes a consent sub-protocol having a plurality of defined steps to prompt for and receive consent/assent to conduct a medical procedure on a patient.

19. The apparatus as in claim 1 further comprising at least one time display configured to monitor and display time being taken to complete individual or multiple protocol steps.

20. The apparatus as in claim 1 further comprising any one of or a combination of at least one temperature sensor, pressure sensor, vibration sensor, location sensor, and movement sensor and wherein the processor is configured to receive and store data from each sensor.

21. The apparatus as in claim 1 further comprising a timer configured to provide a visual and/or audio output of time for at least one step of a study protocol.

22. The apparatus as in claim 1 wherein the compartment is configured for connection to an emergency vehicle having a pre-determined volume and weight limit for the compartment.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
